(19) United States
(12) Patent Application Publication
(10) Pub. No.: US 2018/0053140 A1
(43) Pub. Date: Feb. 22, 2018

(54) FOOD STORAGE METHOD AND APPARATUS WITH SENSORS

(71) Applicant: Intel Corporation, Santa Clara, CA (US)

(72) Inventors: Jim S. Baca, Corrales, NM (US); James G. Simmons, Phoenix, AZ (US); David Stanasolovich, Hillsboro, OR (US); David W. Baker, Gilbert, AZ (US)

(21) Appl. No.: 15/240,882

(22) Filed: Aug. 18, 2016

(51) Int. Cl. G06Q 10/08; G01N 33/02

(52) U.S. Cl. CPC G06Q 10/087; G01N 33/02

(57) ABSTRACT

The present disclosure describes a number of embodiments related to devices, systems, and methods for storing food that may include one or more sensors to collect sensor data associated with one or more food items, and one or more computer processors running a food condition module communicatively coupled to the plurality of sensors to determine a condition of the one or more food items by processing the sensor data to identify the one or more food items and one or more attributes of the one or more food items, and determine a condition of the one or more food items based on the one or more attributes of the one or more food items, and menu opportunity and restocking modules to aid the efficient and intelligent consumption of food.

### FIELD

Embodiments of the present disclosure generally relate to the field of food storage and monitoring. More specifically, embodiments of the present disclosure relate to devices and methods for sensing food items, food condition, and so forth.

### BACKGROUND

Food wastage is a pervasive problem. Food wastage typically occurs when food is not consumed before it expires or spoils. This results in environmental losses, monetary losses, and time losses as people spend additional time at the grocery store and other retail food outlets to replace their food items. Wastage frequently occurs as the food is stored in a home or business, such as in a refrigerator, a pantry and/or a food cupboard. In addition to losses that may be personal to an individual or a business, food wastage drives up costs for everybody creating a false increase demand for food. In legacy implementations, food wastage is typically tracked by manually checking food expiration dates and visually inspecting produce at regular intervals.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 is a diagram of intelligent food storage with sensors, in accordance with various embodiments.

FIG. 2 is a diagram of relationships between intelligent food storage and client devices, in accordance with various embodiments.

FIG. 3 illustrates interactions of an intelligent food consumption (IFC) module with a user, in accordance with various embodiments.

FIG. 4 illustrates an example computing system suitable for practicing various aspects of the disclosure, in accordance with various embodiments.

FIG. 5 is a block diagram illustrates a method for implementing intelligent food storage, in accordance with various embodiments.

FIG. 6 is a diagram illustrating computer readable media having instructions for practicing intelligent food storage, in accordance with various embodiments.

### DETAILED DESCRIPTION

Processes, apparatuses, and systems for intelligent food storage and consumption, are disclosed herein.

In embodiments, an apparatus for storing food may be disclosed that includes one or more sensors to collect sensor data associated with one or more food items, and one or more computer processors running a food condition module communicatively coupled to the plurality of sensors to determine a condition of the one or more food items by processing the sensor data to identify the one or more food items and one or more attributes of the one or more food items, and determine a condition of the one or more food items based on the one or more attributes of the one or more food items.

In embodiments, the apparatus may use sensors such as three-dimensional depth-based cameras and odor sensors along with predictive analytics with real-time learning into an intelligent food consumption platform, for example for a refrigerator and/or a pantry, to know exactly when certain foods will expire, and what foods may need to be replaced and when. In embodiments, an inventory tracking system that may suggest possible meals to prepare based on the inventory. Advantages of such an apparatus, or of their associated processes may include less food waste, creative menu planning based on most efficient use of on-hand food items based on their condition, less time wasted going to a grocery store to replace food items, and preventing spoiled food from being consumed.

Details of these and/or other embodiments, as well as some advantages and benefits, are disclosed and described herein.

In the following description, various aspects of the illustrative implementations are described using terms commonly employed by those skilled in the art to convey the substance of their work to others skilled in the art. However, it will be apparent to those skilled in the art that embodiments of the present disclosure may be practiced with only some of the described aspects. For purposes of explanation, specific numbers, materials, and configurations are set forth in order to provide a thorough understanding of the illustrative implementations. However, it will be apparent to one skilled in the art that embodiments of the present disclosure may be practiced without the specific details. In other instances, well-known features are omitted or simplified in order not to obscure the illustrative implementations.

In the following description, reference is made to the accompanying drawings that form a part hereof, wherein like numerals designate like parts throughout, and in which is shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

The description may use perspective-based descriptions such as top/bottom, in/out, over/under, and the like. Such descriptions are merely used to facilitate the discussion and are not intended to restrict the application of embodiments described herein to any particular orientation.

The description may use the phrases “in an embodiment,” or “in embodiments,” which may each refer to one or more of the same or different embodiments. Furthermore, the terms “including,” “having,” and the like, as used with respect to embodiments of the present disclosure, are synonymous. As used herein, the term “module” may refer to, be part of, or include an Application Specific Integrated Circuit (ASIC), an electronic circuit, a processor (shared, dedicated, or group) and/or memory (shared, dedicated, or group) that execute one or more software or firmware programs having machine instructions (generated from an assembler or compiled from a high level language compiler), a combinational logic circuit, and/or other suitable components that provide the described functionality.

The terms “coupled with” and “coupled to” and the like may be used herein. “Coupled” may mean one or more of the following. “Coupled” may mean that two or more elements are in direct physical or electrical contact. However, “coupled” may also mean that two or more elements indirectly contact each other, but yet still cooperate or interact with each other, and may mean that one or more other elements are coupled or connected between the elements that are said to be coupled with each other. By way of example and not limitation, “coupled” may mean two or more elements or devices are coupled by electrical connections on a printed circuit board such as a motherboard, for example. By way of example and not limitation, “coupled” may mean two or more elements/devices cooperate and/or interact through one or more network linkages such as wired and/or wireless networks. By way of example and not limitation, a computing apparatus may include two or more computing devices “coupled” on a motherboard or by one or more network linkages.

Various operations are described as multiple discrete operations in turn, in a manner that is most helpful in understanding the claimed subject matter. However, the order of description should not be construed as to imply that these operations are necessarily order dependent.

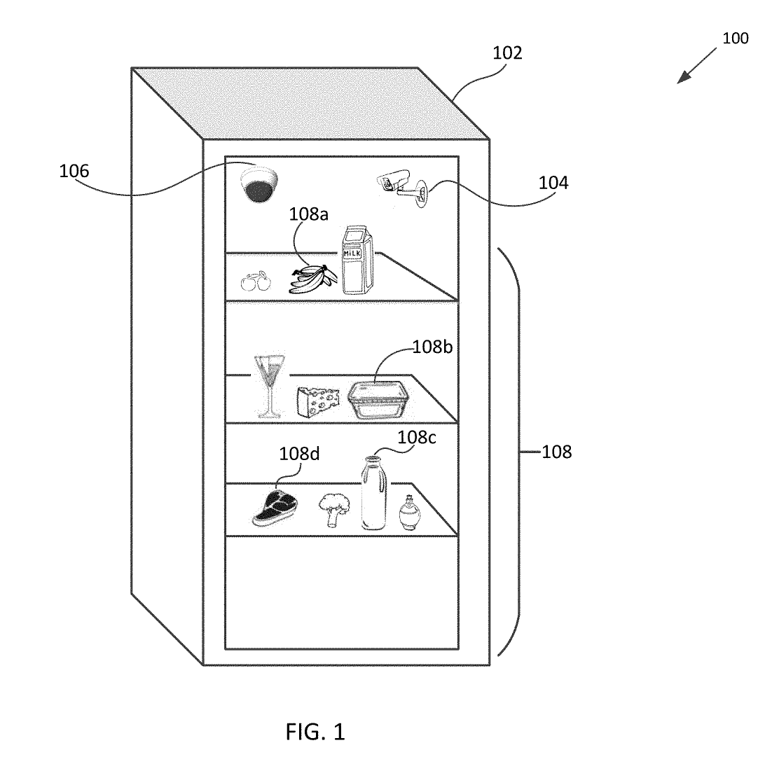

FIG. 1 is a diagram of an example intelligent food storage with sensors, in accordance with various embodiments. Diagram 100 shows a food storage 102 that may be used to store various food items 108. The food storage 102 may contain multiple sensors, for example, but not limited to, a video camera 104, or ambient air chemical detector(s) 106 to collect and generate sensor data associated with food items in food storage 102. In embodiments, other sensors, e.g., electronic, thermal, tactile, so forth, that detect other conditions, such as temperature, humidity, softness/stiffness, RFIDs, barcodes, and so forth, may be used, and the sensors may be in multiple locations within food storage 102. In embodiments, the food storage 102 may have sub-compartments that may contain additional sensors that may be specific to those sub-compartments (not shown).

In embodiments, the sensors may be used to generate sensor data to facilitate identification of food items 108 that are within food storage 102. In embodiments, the sensor data may be used to determine the condition of the food items 108 by sensing various attributes of the food items 108. In embodiments, a condition of a food item 108 may vary according to the type of the food item 108. In one non-limiting example, the condition of a produce-type food item, such as a banana 108a, may include under ripe (such as a green banana), ripe (such as a yellow banana), overripe (such as a black spotted banana), spoiled (such as a black banana), rotting (such as a mushy black banana with mold growing on it), and the like. A particular food item may be suitable for a variety of different modes of consumption when the food item is in a number of different conditions. For example, a ripe banana may be suitable for eating raw and an overripe banana not suitable; however, an overripe banana may be suitable for making banana bread and a ripe banana not suitable.

Continuing with the example of the banana 108a, a number of sensors may be used to collect sensor data to be used to determine the condition of a banana within food storage 102. A camera 104 which may be, for example, a 3-D camera with RealSense™ technology, may be used to capture a 3-D image that may be used identify the object as a banana. For example, analysis of the color of the image may provide an indication of the bananas ripeness. Movement of the banana captured by multiple images over time may indicate a softening of the banana that may also indicate banana ripeness. Odor and/or chemical sensor data, and sets of these data captured over time may indicate increased level of ethylene gas that may be produced by a ripening banana to indicate the banana's condition (such as ripe, overripe, or spoiled). Thermal sensors may be used to identify increased spots of heat within the banana that may indicate areas of beginning spoilage. Tactile sensors or other sensors may be used to record data of density or softness of different areas of the banana that may indicate a level of ripeness. The camera may also include metadata of the captured image that may include the time the image was captured, the color depth of the image, and so forth, may also be used to identify information about the banana.

In other non-limiting examples of food items, for example cheese, data from the sensors may be used to identify levels of aging to indicate when the cheese may be ready for consumption.

In other non-limiting examples, sensor data collected by sensors within food storage 102 may also be used to identify and to provide data on leftover food items. In a non-limiting example, sensor data may be used to identify and to provide data on a leftover dish of rigatoni 108b, that may be used to assess the freshness or safety of consuming the rigatoni 108b. In addition to the activity of sensors described above, images from the camera 104 may be used to identify writing on a container or other indications of a date by which the rigatoni 108b should be consumed.

In other non-limiting examples, images from the camera 104 may help identify expiration dates present on food items in commercial containers or wrapping, such as a jar or a bottle item 108c, or a meat item 108d. In addition, other sensors may be used that interact with food items or the containers in which food items are kept, for example to identify barcodes or to receive data from RFID tags.

In embodiments, food storage 102 may be sealed. In non-limiting examples, this may include a refrigerator, freezer, or cargo container. In embodiments, food storage 102 may be partially sealed, for example, a pantry or a cooler. In embodiments, such food storage 102 may be open, for example a shelf or a counter.

In embodiments, as described earlier, a sensor may include a camera, a three-dimensional camera, or an infrared-based camera. For example, a 3-D camera with RealSense™ Technology able to take photographs that contain three dimensional depth/disparity information in a video or other picture file that may be analyzed to determine what food items 108 may be in food storage 102, as well as attributes of the various food items 108.

In other embodiments, as described earlier, a sensor may include a thermometer, a humidity detector, a chemical analysis sensor, or an odor detector. In embodiments, a sensor may include a tactile sensor or ultrasonic sensor that may collect sensor data to identify the surface texture or softness of an item of food 108. These sensors may be used to provide data for predictive analysis that may be used to determine when a food item in food storage 102 such as a refrigerator or pantry is either set to expire or is approaching spoilage. In embodiments, sensor data from the sensors in food storage 102 may be used to separate the food items 108, in real time, into food types and food characteristics. In non-limiting examples, sensor data from the sensors may be used to determine that lettuce edges are brown or that dark spots appear on fruit, which then may be delivered to a predictive analytic system to determine that the lettuce or the fruit is about to be spoiled.

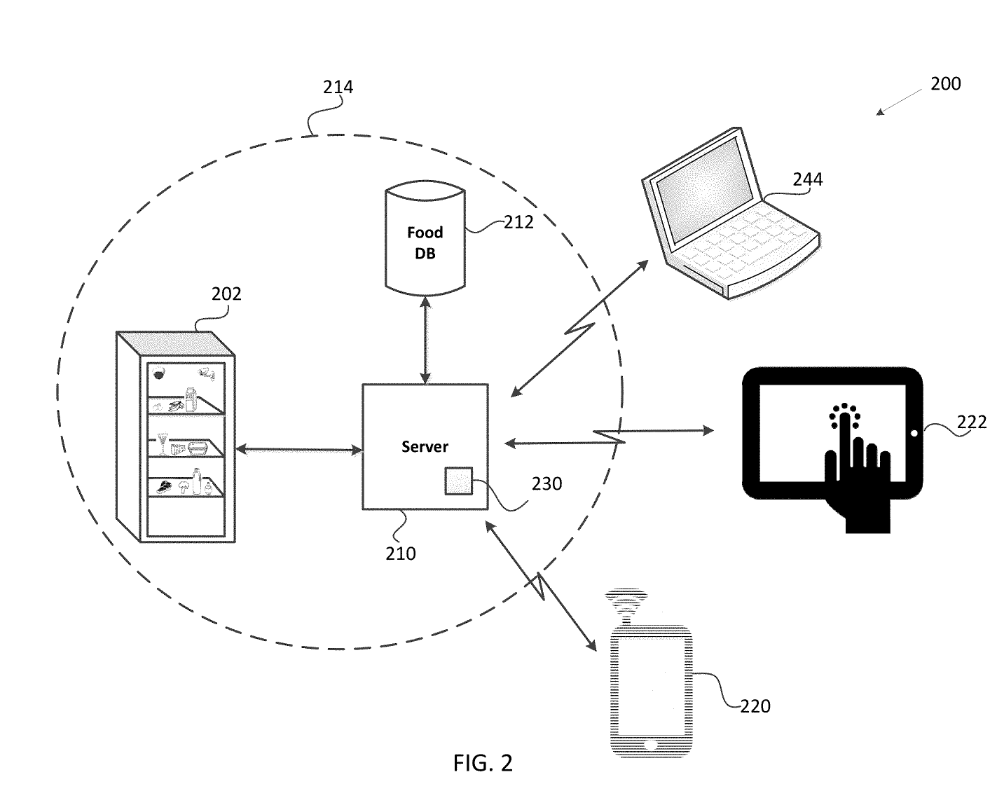

FIG. 2 is a diagram of example relationships between intelligent food storage and client devices, in accordance with various embodiments. Diagram 200 shows food storage 202, which may be similar to food storage 102 of FIG. 1, communicatively coupled with a server 210. In embodiments, the server 210 may receive sensor data from sensors included in the food storage 202, which may be similar to sensors 104, 106 of FIG. 1. The database 212 may be coupled with the server 210 (and in some embodiments, with the food storage 202). The database 212 may store sensor data received from the sensors within food storage 202, and may also store information generated by server 210 during sensor data analysis and processing. In embodiments, the functions of the server 210 and/or the functions of the database 212 may be incorporated into food storage apparatus 202, or may be separate from food storage apparatus 202 (as shown). In embodiments, food storage 202, the server 210, and database 212 may be referred to as an intelligent food consumption (IFC) platform 214.

In embodiments, various user devices may be used to communicate with the IFC platform 214. These devices may include smartphones 220, tablets 222, or personal computers 224. Other devices, such as other computing servers or other data aggregation points (not shown) may also communicate with the IFC platform 214. One example of such a device may be an automated ordering system that is connected to a grocery vendor.

In embodiments, the server 210 may include predictive analytics functions and/or real-time learning functions to provide useful information and/or recommendations for actions based upon sensor data received from the sensors included in the food storage 202. In embodiments, predictive analytics may be based on pattern recognition models such as a hidden Markov model, to identify the food items 108 within food storage 202, the condition of the individual food items within food storage 202, and to determine if a food item is fresh, spoiled, calculate/predict an estimated time to spoilage, or perform some other prediction of the future condition of the food item.

In embodiments, the IFC platform 214 may also be configured to identify and/or store when individual food items have been added to or have been taken from the food storage 202. This information may then be combined with determinations made by the server 210 to identify when the food storage 202 may be running out of certain food items, in addition to those food items which may be expired or may be about to expire, to identify inventory levels or to identify when to restock food items.

In embodiments, server 210 may include an IFC module 230 that may communicate with the client devices 220, 222 and 244 to provide information and/or insight into the food items 108 condition within food storage 202 and to a user. In examples, the IFC module 230 may provide information, for example, on food freshness, consumption patterns, menu opportunities, and the like. In embodiments, IFC module 230 may be an ASIC, an electronic circuit, a processor (shared, dedicated, or group) and/or memory (shared, dedicated, or group) that execute one or more software or firmware programs having machine instructions (generated from an assembler or compiled from a high level language compiler), and/or a combinational logic circuit with programmed logic to provide the described functions.

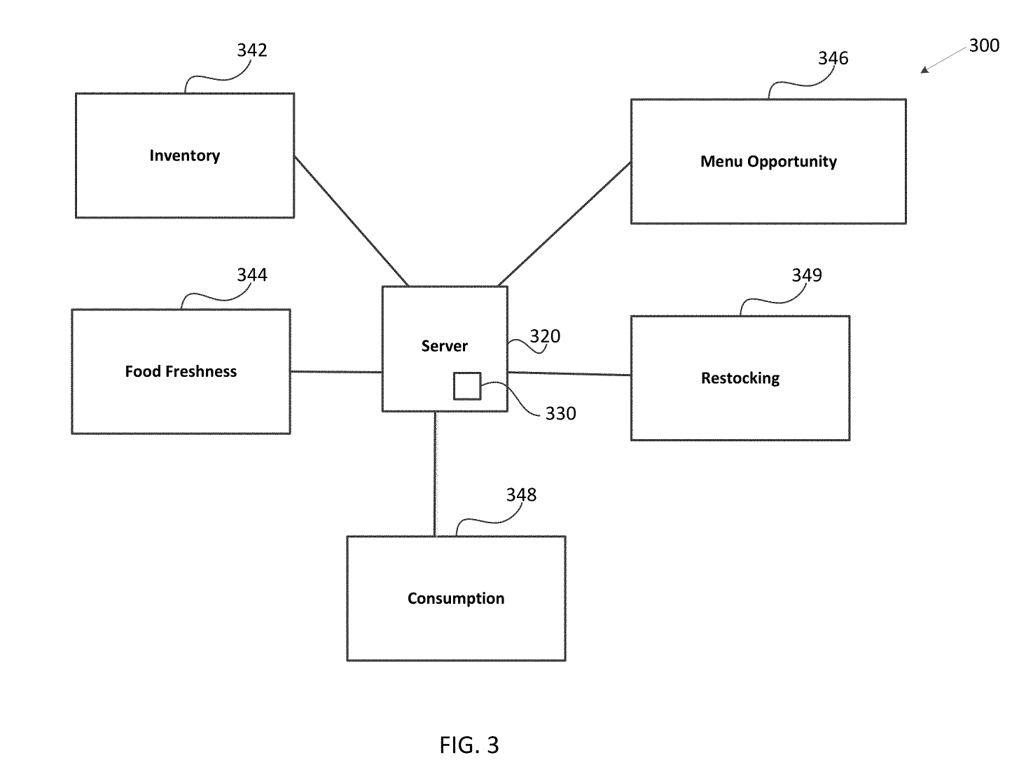

FIG. 3 illustrates example services of an IFC module, in accordance with various embodiments. Diagram 300 shows an IFC module 330, which may be similar to the IFC module 230 of FIG. 2. In embodiments, the IFC module 330 may be a component of a server 310, which may be similar to server 210 of FIG. 2, and communicate, e.g., wirelessly, with the client devices 220, 222 and 244 of FIG. 2. In embodiments, a portion of the IFC module 330 may be part of a client device, such as smartphone 220, while another portion be part of the IFC platform 214, with the two portions communicatively coupled. In embodiments where the IFC module 330 is entirely part of the IFC platform 214, for example, part of server 210, the client devices 220, 222 and 244 may interact with the IFC module 330 via e.g., a generic agent, such as a browser.

In embodiments, the IFC module 330 may include circuitry and/or instructions that implement or facilitate an inventory function 342. In embodiments, the IFC module 330 may receive/retrieve data collected by the sensors in real time, or stored in database 212. The inventory function 342 may then determine the complete inventory of food items 108 in the food storage 220. In embodiments, the inventory may be broken down into food items on hand that are (1) ready to be consumed, (2) not ready to be consumed because they are not ripe, and/or (3) should not be consumed because they are spoiled and/or expired, and should be thrown out.

In embodiments, the IFC module 330 may include circuitry and/or instructions that implement or facilitate a menu opportunity function 346. In embodiments, this function may indicate to a user what food items 108 are available to eat, or what menus or meals may be made with a combination of the food items 108 available to eat. In one non-limiting example, a user may open a generic agent or an agent of IFC module 330 on his smartphone 220 to interact with menu opportunity function 346 to determine menu opportunities. Menu opportunities may be based on entirely on food items on hand, or supplemented by items if the user were to stop at the grocery store. The menu opportunity function 346 may acquire the inventory information from inventory function 342, and/or freshness information from food freshness function 344.

In embodiments, the IFC module 330 may include circuitry and/or instructions that implement a food freshness function 344 that provides information regarding food freshness. The user may use the latest freshness information for the food items 108 to manage his food storage 102, for example his refrigerator and/or his pantry. The food freshness function 344 may report to the user that leftover rigatoni from the weekend will be fresh through tomorrow and the green beans and tomatoes may be eaten for up to another 48 hours, but that the salad is starting to brown and should not be eaten. The food freshness function 344 and menu opportunity function 346 together may then suggest to the user a meal of rigatoni, green beans, and tomatoes to consume these items before expiration. In embodiments, a number of possible menu items may be presented that use food items 108 that are to expire soon. In embodiments, menu items may change based upon the condition of one or more food items 108. In a non-limiting example, if an apple is under-ripe, the menu opportunity function 346 may recommend grinding the apple with cranberries to create a sauce for lamb. However, if the apple is ripe or over-ripe, the menu opportunity function 346 may recommend juicing the apple.

In embodiments, food freshness 344 may include a general condition of one or more food items 108. In embodiments, the food freshness function 344, in addition to providing condition information, may also allow users to view their current food items 108 in the food store 102. In non-limiting examples, the food freshness function 344 may display on client device 220, 222 or 244 a live video of the food items 108 in a refrigerator, and may also display the food real-time freshness data and (in conjunction with inventory function 342) current inventory, to give users additional information to determine if they need to purchase one or more additional food items.

In embodiments, the IFC module 330 may include circuitry and/or instructions that implement a restocking function 349 for food storage 202. In embodiments that may involve residential and/or home use, a user may have a difficult time keeping on top of contents of the many food items 108 in his pantry and refrigerator. In practice, he may continuously run out of his favorite and special use foods while other foods expire or become spoiled and have to be thrown out. The user may frequently go to the grocery store but may not be aware that certain items have expired, spoiled or are depleted. In embodiments, the restocking function 349 (in cooperation with inventory function 342 and food freshness function 344) may determine which food items like lettuce, pasta sauce, milk, and the like that have expired or are close to spoiling. The restocking function 349 may also determine the list of non-food items that may need to be repurchased. The restocking function 349 may then provide a real-time updated list of in-stock food items as well as non-food items and a list of items that should be purchased within a certain amount of time. In a non-limiting example, the restocking function 349 may then send a real-time updated list to the user's client device 220, 222 and/or 244 indicating that he needs to buy milk today, lettuce tomorrow, and pasta sauce within three days. In this way, IFC module 330 may utilize the contextual 3D and sensor data to manage the inventory of food items in their refrigerator and pantry, while integrating the real time freshness and expiration data to accurately identify what food items 108 need to be purchased.

In embodiments, the IFC module 330 may be used to manage a food supply 108 in commercial environments. For example, the lead chef in a popular restaurant may have many food items which are in high demand, and some food items which may intermittently be in high demand. In addition, there may be times when the chef runs out of some ingredients while other ingredients are spoiled and must be discarded. Using the IFC module 330, the chef and her supply manager may use the IFC module 330 to manage the freshness and inventory of their food items in real-time. In addition, her inventory system may also be connected with her vendors' ordering systems so that food items are ordered and timely delivered. This may result in both food shortages in the hotel and food waste from spoilage to be significantly reduced. In addition, the IFC module 330 may be used to assist the chef by identifying freshness levels of various ingredients among the food supply 108. In addition, based on the identified freshness levels of ingredients, specific menu items may be recommended to be created and presented for consumption. For example, a restaurant may use these recommended menu items to generate daily specials in order to encourage restaurant patrons to order menu items that will consume ingredients from the food supply 108 based on freshness levels of various ingredients.

In embodiments, the restocking function 349 may be implemented as a combination of the inventory function 342 and a real-time shopping list may be created and presented to the user. In one non-limiting example, Kyle's Greek Yogurt consumption and spoilage predicts that he needs to replenish his supply of 10 containers every 3 days for Salted Caramel Greek Yogurt, and every 5 days for Vanilla Greek Yogurt. This information is sent to the real time shopping list on the user's smartphone.

In embodiments, the IFC module 330 may include circuitry and/or instructions that implement or facilitate a consumption function 348. In embodiments, a consumption function 348 (in cooperation with inventory function 342 and/or food freshness function 344) may determine changes in food storage 202 inventory over periods of time, and whether the food items 108 were consumed, or had to be thrown out because they were spoiled. In addition, information on the nutritional value and of the types and amounts of food items 108 consumed over a period of time may also be provided. In addition, historic spoilage rates by particular types of food may be identified and presented to user.

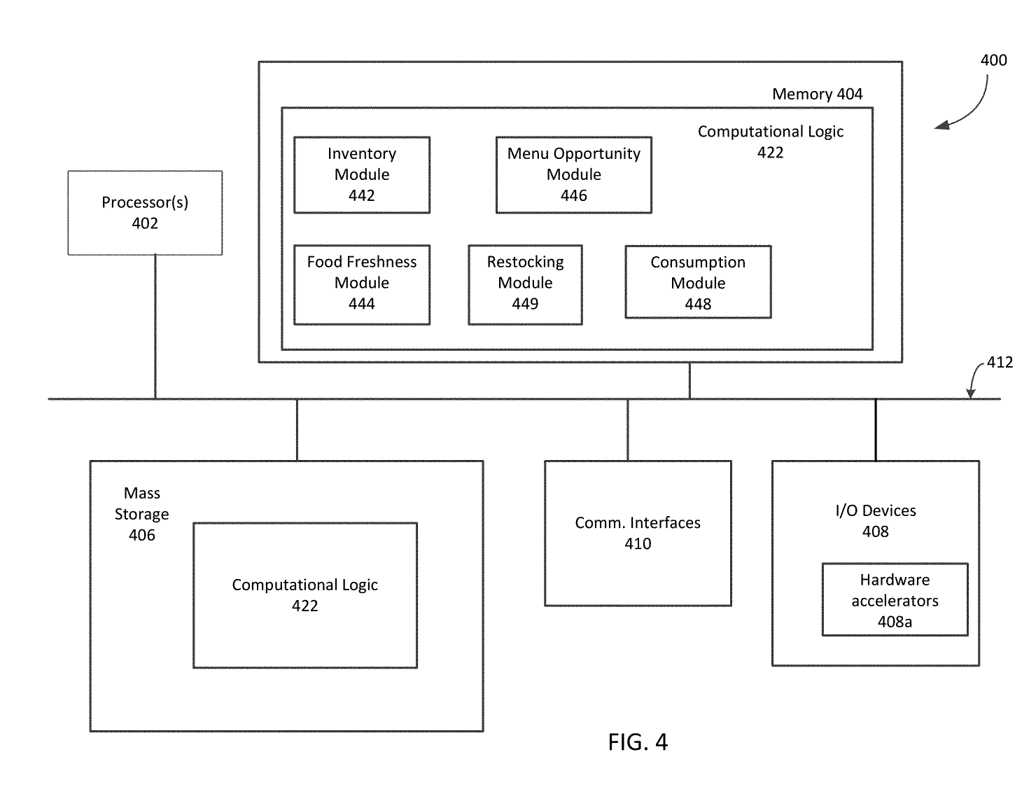

FIG. 4 illustrates an example computing device 400 and may be suitable for use to practice aspects of the present disclosure, in accordance with various embodiments. For example, the example computing device 400 may be suitable to implement the functionalities associated with diagrams 202, 210, 212, 214, 300, 342, 344, 346, 348, 349, 502, 504, and/or 506.

As shown, computing device 400 may include one or more processors 402, each having one or more processor cores, and system memory 404. The processor 402 may include any type of unicore or multi-core processors. Each processor core may include a central processing unit (CPU), and one or more level of caches. The processor 402 may be implemented as an integrated circuit. The computing device 400 may include mass storage devices 406 (such as diskette, hard drive, volatile memory (e.g., dynamic random access memory (DRAM)), compact disc read only memory (CD-ROM), digital versatile disk (DVD) and so forth). In general, system memory 404 and/or mass storage devices 406 may be temporal and/or persistent storage of any type, including, but not limited to, volatile and non-volatile memory, optical, magnetic, and/or solid state mass storage, and so forth. Volatile memory may include, but not be limited to, static and/or dynamic random access memory. Non-volatile memory may include, but not be limited to, electrically erasable programmable read only memory, phase change memory, resistive memory, and so forth. In embodiments, the mass storage device 406 may be used to implement the database 212 of FIG. 2.

The computing device 400 may further include input/output (I/O) devices 408 such as the earlier described sensors. In embodiments, I/O devices 408 may include optional hardware accelerators 408a to accelerate e.g., initial processing of the sensor data. In embodiments, I/O devices 408 may further include a display, keyboard, cursor control, remote control, gaming controller, image capture device, one or more three-dimensional cameras used to capture images, and so forth, and communication interfaces 410 (such as network interface cards, modems, infrared receivers, radio receivers (e.g., Bluetooth), and so forth). In embodiments, I/O devices 408 may be suitable for communicative connections with three-dimensional cameras, sensors, or user devices instead. In some embodiments, I/O devices 408 when used as user devices may include devices necessary for implementing the functionalities of receiving an image captured by a camera such as camera 104, or receiving information from sensors 106 as described in reference to FIG. 1. In embodiments, some of the optional hardware accelerators may be disposed with communication interfaces 410 instead.

The communication interfaces 410 may include communication chips (not shown) that may be configured to operate the device 400 in accordance with a Global System for Mobile Communication (GSM), General Packet Radio Service (GPRS), Universal Mobile Telecommunications System (UMTS), High Speed Packet Access (HSPA), Evolved HSPA (E-HSPA), or Long Term Evolution (LTE) network. The communication chips may also be configured to operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chips may be configured to operate in accordance with Code Division Multiple Access (CDMA), Time Division Multiple Access (TDMA), Digital Enhanced Cordless Telecommunications (DECT), Evolution-Data Optimized (EV-DO), derivatives thereof, as well as any other wireless protocols that are designated as 3G, 4G, 5G, and beyond. The communication interfaces 410 may operate in accordance with other wireless protocols in other embodiments.

The above-described computing device 400 elements may be coupled to each other via system bus 412, which may represent one or more buses. In the case of multiple buses, they may be bridged by one or more bus bridges (not shown). Each of these elements may perform its conventional functions known in the art. In particular, system memory 404 and mass storage devices 406 may be employed to store a working copy and a permanent copy of the programming instructions implementing the operations and functionalities associated with diagrams 202, 210, 212, 214, 300, 342, 344, 346, 348, 349, 502, 504, and/or 506, generally shown as computational logic 422. Computational logic 422 may be implemented by assembler instructions supported by processor(s) 402 or high-level languages that may be compiled into such instructions.

In embodiments, the Computational Logic 422 may contain a food condition module 450, which may perform one or more of the functions associated with FIG. 2 diagram 214, or with FIGS. 2-6. Computational Logic 422 may contain an inventory module 442, food freshness module 444, menu opportunity module 446, restocking module 449 and consumption module of FIGS. 2-6. In embodiments, part or all of the computational logic 422 may be implemented in a mobile computing device, such as a smart phone 220, tablet 222, and/or personal computer 244.

The permanent copy of the programming instructions may be placed into mass storage devices 406 in the factory, or in the field, though, for example, a distribution medium (not shown), such as a compact disc (CD), or through communication interfaces 410 (from a distribution server (not shown)).

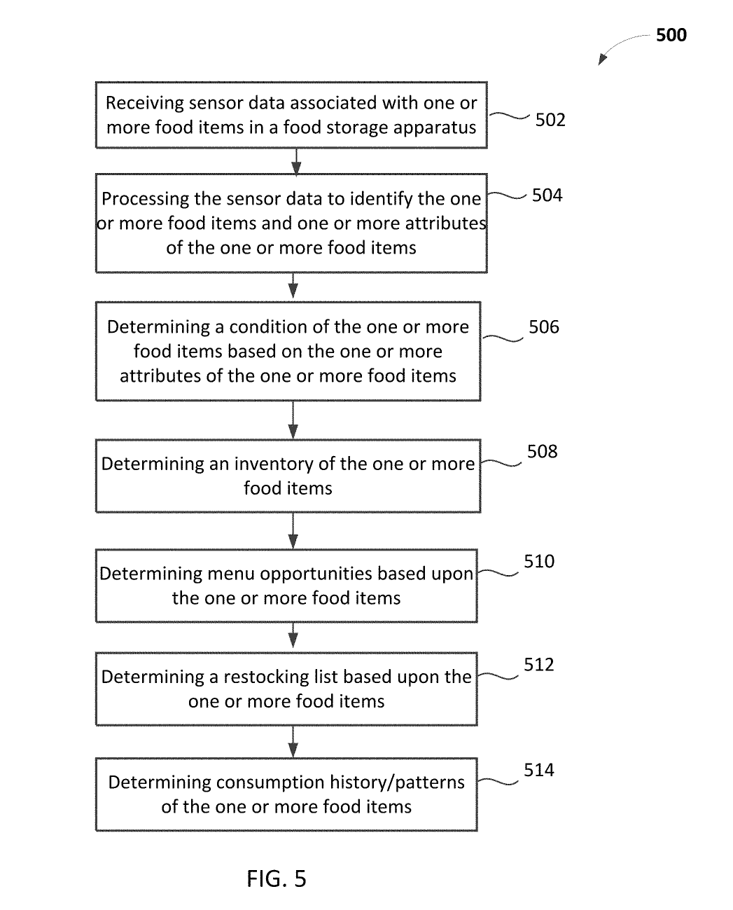

FIG. 5 is a block diagram 500 that illustrates a method for implementing food storage for intelligent consumption, in accordance with various embodiments. In some embodiments, the server 210 and/or the IFC platform 214 of FIG. 2 may perform one or more processes, such as the process 500. In some embodiments, the operations performed at the various blocks may be divided, combined, omitted, and/or in different order.

At block 502, the process may receive sensor data associated with one or more food items in a food storage apparatus. In embodiments, this sensor data may come from one or more sensors within a food storage 102, for example camera 104 or sensor 106 of FIG. 1.

At block 504, the process may process the sensor data to identify one or more food items and one or more attributes of the one or more food items. In embodiments, the processing may be done by the server 210. In embodiments, the processing may involve access to the database 212 that may contain previous sensor data from food items in food storage 202.

At block 506, the process may determine a condition/freshness of the one or more food items based on the one or more attributes of the one or more food items.

At block 508, the process may further determine an inventory of the one or more food items. In embodiments, this may include using sensor data to identify and to count the items within food storage 102.

At block 510, the process may further determine menu opportunities based upon the one or more food items. In embodiments, menus, which may include combinations of ingredients use to make an item on the menu, may be known. In embodiments, the process may determine which of the menu items may be made based upon the items within food storage 102, and considered these menu items as menu opportunities.

At block 512, the process may further determine a restocking list based upon the one or more food items. In embodiments, restocking this may be based upon an analysis of the food items 108, consumption history and/or consumption patterns of the food items 108, and whether any of the food items 108 are expired and/or spoiled. In embodiments, a stocking list may be presented to a user, for example on a smart phone 220, or may be sent directly to a vendor for automatic ordering and delivery to the food storage 102.

At block 514, the process may further determine consumption history/patterns of the one or more food items. In embodiments, history/patterns of food consumption may be based upon inventories of a food storage 102 overtime to determine how the food items 108 change over time. In embodiments, the regularity of restocking of the food storage 102 may also be considered in determining consumption history.

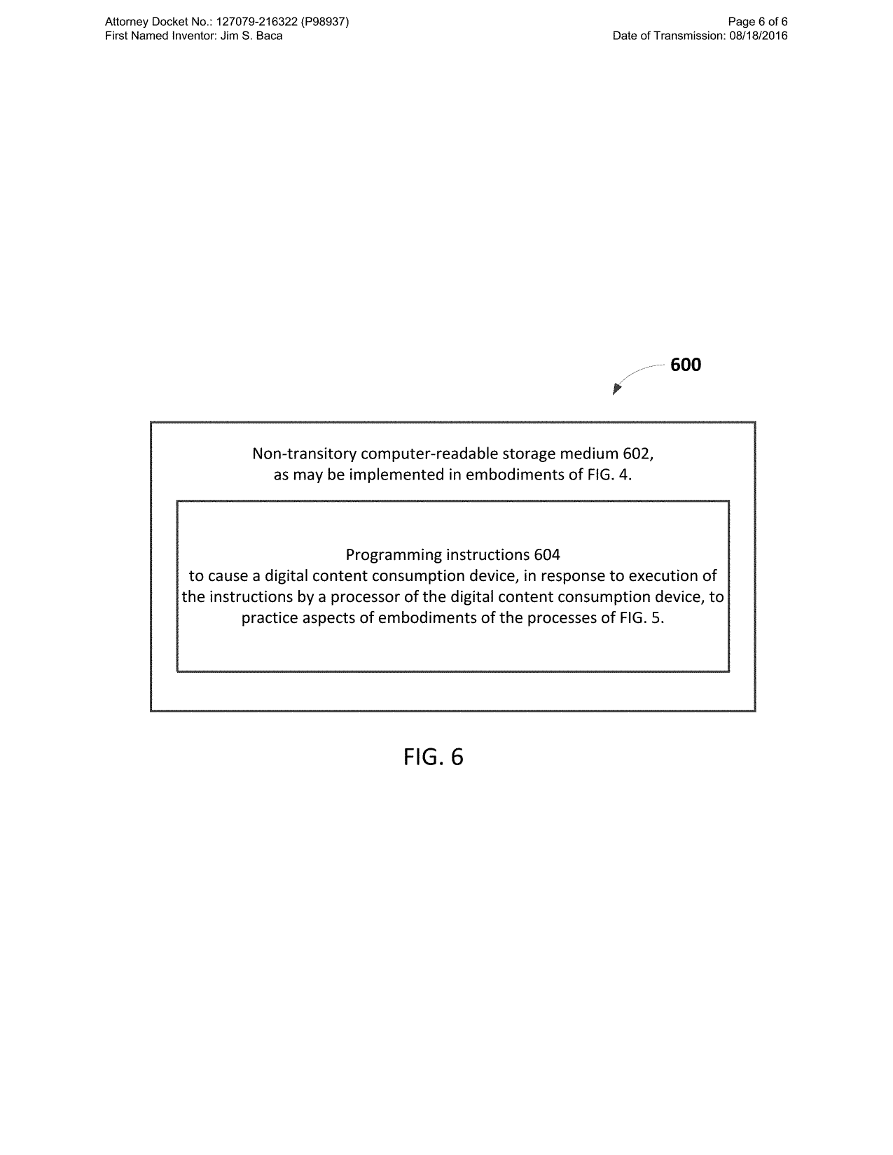

FIG. 6 is a diagram 600 illustrating computer readable media having instructions for practicing the above-described techniques, or for programming/causing systems and devices to perform the above-described techniques, in accordance with various embodiments. In some embodiments, such computer readable media 602 may be included in a memory or storage device, which may be transitory or non-transitory, of the intelligent food consumption platform 214 of FIG. 2. In embodiments, instructions 604, in response to execution by a processor of computing device may cause the computing device to implement the inventory function 342, the food freshness function 344, the menu opportunity function 346, the re-stocking function 349, and/or the consumption function 348. In embodiments, instructions 604 may include assembler instructions supported by a processing device, or may include instructions in a high-level language, such as C, that can be compiled into object code executable by the processing device. In some embodiments, a persistent copy of the computer readable instructions 604 may be placed into a persistent storage device in the factory or in the field (through, for example, a machine-accessible distribution medium (not shown)). In some embodiments, a persistent copy of the computer readable instructions 604 may be placed into a persistent storage device through a suitable communication pathway (e.g., from a distribution server).

The corresponding structures, material, acts, and equivalents of all means or steps plus function elements in the claims below are intended to include any structure, material or act for performing the function in combination with other claimed elements are specifically claimed. The description of the present disclosure has been presented for purposes of illustration and description, but is not intended to be exhaustive or limited to the disclosure in the form disclosed. Many modifications and variations will be apparent to those of ordinary skill without departing from the scope and spirit of the disclosure. The embodiment was chosen and described in order to best explain the principles of the disclosure and the practical application, and to enable others of ordinary skill in the art to understand the disclosure for embodiments with various modifications as are suited to the particular use contemplated.

### Examples

Examples, according to various embodiments, may include the following.

Example 1 may be an apparatus for storing food, comprising: one or more sensors to collect sensor data associated with one or more food items; one or more computer processors; and a food condition module communicatively coupled to the one or more processors and the plurality of sensors to determine condition of the one or more food items; wherein to determine, the food condition module is to: process the sensor data to identify the one or more food items and one or more attributes of the one or more food items, and determine a condition of the one or more food items based on the one or more attributes of the one or more food items.

Example 2 may include the subject matter of Example 1, wherein the food condition module is to further store, in a database, the determined condition of the one or more food items.

Example 3 may include the subject matter of any Examples 1-2, wherein the apparatus further includes: a food recommendation module communicatively coupled to the one or more processors to provide a recommendation with respect to the identified one or more food items; wherein to provide a recommendation, the food recommendation module is to: receive a request for a recommendation; determine, based upon the received request and the condition of the one or more food items, a recommendation; and transmit the determined recommendation to a recipient.

Example 4 may include the subject matter of Example 3, wherein the recommendation is one or more of: which of the one or more food items to throw away; which of the one or more food items should be consumed within a first defined time period; which of the one or more food items should be consumed after a second defined time period; which of the one or more food items may be consumed within a third defined time period; which of the one or more food items should be sent to a first destination by a first date; or a best time to consume the one or more item of food.

Example 5 may include the subject matter of Example 3, wherein the recommendation is one or more of: what recipes may be made using the one or more food items; or what meals may be prepared with the one or more food items.

Example 6 may include the subject matter of Example 3, wherein the recommendation is which food items should be replenished and at what time.

Example 7 may include the subject matter of Example 2, wherein to determine a recommendation is further to: compare the condition of the one or more food items with the stored condition of the one or more food items in the database.

Example 8 may include the subject matter of Example 7, wherein the recommendation is an indication of consumed food, based upon the comparison.

Example 9 may include the subject matter of any Examples 3-8, wherein to determine a recommendation is further to determine a recommendation based on a predictive analytics engine.

Example 10 may include the subject matter of Example 3, wherein transmit the determined recommendation further comprises: transmit or cause to transmit a request for an action, the action based upon the determined recommendation.

Example 11 may include the subject matter of Example 1, wherein a sensor is a camera, a 3D camera, or an infrared camera.

Example 12 may include the subject matter of Example 1, wherein a sensor is a thermometer, a humidity detector, a chemical analysis sensor, or an odor detector.

Example 13 may include the subject matter of Example 1, wherein a sensor is a mass sensor or a density sensor.

Example 14 may include the subject matter of Example 1, wherein a sensor is a softness sensor

Example 15 may include the subject matter of any Examples 1-14, wherein the apparatus is a refrigerator, a freezer, a shipping container, a cooler, a pantry or a warehouse storage space.

Example 16 may be a method for storing food using a food storage apparatus comprising: receiving, by the apparatus, sensor data associated with one or more food items in the food storage apparatus; processing, by the apparatus, the sensor data to identify the one or more food items and one or more attributes of the one or more food items; and determining, by the apparatus, an inventory of the one or more food items based on the one or more attributes of the one or more food items.

Example 17 may include the subject matter of Example 16, further comprising storing, by the apparatus, in a database, the determined inventory.

Example 18 may include the method of any of Examples 16-17, further comprising: receiving, by the apparatus, a request for a recommendation; determining, by the apparatus, based upon the received request and the inventory of the one or more food items, a recommendation; and transmitting, by the apparatus, the determined recommendation to a recipient.

Example 19 may include the subject matter of Example 18, wherein the recommendation is one or more of: which of the one or more food items to throw away; which of the one or more food items should be consumed within a first defined time period; which of the one or more food items should be consumed after a second defined time period; which of the one or more food items may be consumed within a third defined time period; which of the one or more food items should be sent to a first destination by a first date; or a best time to consume the one or more item of food.

Example 20 may include the subject matter of Example 18, wherein the recommendation is one or more of: what recipes may be made using the one or more food items; or what meals may be prepared with the one or more food items.

Example 21 may include the subject matter of Example 18, wherein the recommendation is which food items should be replenished and at what time.

Example 22 may include the subject matter of Example 18, wherein determining a recommendation further comprises: comparing, by the apparatus, the inventory of the one or more food items with the stored inventory of the one or more food items in the database.

Example 23 may include the subject matter of Example 22, wherein the recommendation is an indication of food items that have been consumed or are spoiled, based upon the comparison.

Example 24 may include the subject matter of any of examples 18-23, wherein determining a recommendation further comprises determining, by the apparatus, a recommendation based on a predictive analytics engine.

Example 25 may include the subject matter of Example 18, wherein transmitting the determined recommendation further comprises transmitting or causing to transmit a request for an action to a recipient, the action based upon the determined recommendation.

Example 26 may include the subject matter of Example 16, wherein a sensor is a camera, a 3D camera, or an infrared camera.

Example 27 may include the subject matter of Example 16, wherein a sensor is a thermometer, a humidity detector, a chemical analysis sensor, or an odor detector.

Example 28 may include the subject matter of Example 16, wherein a sensor is a mass sensor or a density sensor.

Example 29 may include the subject matter of Example 16, wherein a sensor is a softness sensor

Example 30 may include the subject matter of any Examples 16-29, wherein the apparatus is a refrigerator, a freezer, a shipping container, a cooler, a pantry or a warehouse storage space.

Example 31 may be an apparatus for storing food using a food storage apparatus comprising: means for receiving sensor data associated with one or more food items in the food storage apparatus; means for processing the sensor data to identify the one or more food items and one or more attributes of the one or more food items; and means for determining menu opportunities using the one or more food items based on the one or more attributes of the one or more food items.

Example 32 may include the subject matter of Example 31, further comprising storing, by the apparatus, in a database, the determined menu opportunities.

Example 33 may include the subject matter of any Examples 31-32, further comprising: means for receiving a request for a recommendation; means for determining based upon the received request and the menu opportunities of the one or more food items, a recommendation; and means for transmitting the determined recommendation to a recipient.

Example 34 may include the subject matter of Example 33, wherein the recommendation is one or more of: which of the one or more food items to throw away; which of the one or more food items should be consumed within a first defined time period; which of the one or more food items should be consumed after a second defined time period; which of the one or more food items may be consumed within a third defined time period; which of the one or more food items should be sent to a first destination by a first date; or a best time to consume the one or more item of food.

Example 35 may include the subject matter of Example 33, wherein the recommendation is one or more of: what recipes may be made using the one or more food items; or what meals may be prepared with the one or more food items.

Example 36 may include the subject matter of Example 33, wherein the recommendation is which food items should be replenished and at what time.

Example 37 may include the subject matter of Example 33, wherein determining a recommendation further comprises: comparing, by the apparatus, the condition of the one or more food items with the stored condition of the one or more food items in the database.

Example 38 may include the subject matter of Example 37, wherein the recommendation is an indication of food items that have been consumed or are spoiled, based upon the comparison.

Example 39 may include the subject matter of any of Examples 33-38, wherein determining a recommendation further comprises means for determining, by the apparatus, a recommendation based on a predictive analytics engine.

Example 40 may include the subject matter of Example 33, wherein transmitting the determined recommendation further comprises means for transmitting or causing to transmit a request for an action to a recipient, the action based upon the determined recommendation.

Example 41 may include the subject matter of Example 31, wherein a sensor is a camera, a 3D camera, or an infrared camera.

Example 42 may include the subject matter of Example 31, wherein a sensor is a thermometer, a humidity detector, a chemical analysis sensor, or an odor detector.

Example 43 may include the subject matter of Example 31, wherein a sensor is a mass sensor or a density sensor.

Example 44 may include the subject matter of Example 31, wherein a sensor is a softness sensor.

Example 45 may include the subject matter of any Examples 31-44, wherein the apparatus is a refrigerator, a freezer, a shipping container, a cooler, a pantry or a warehouse storage space.

Example 46 may include one or more computer readable media comprising instructions that cause a computing device, in response to execution of the instructions by the computing device, to: receive, by the computing device, sensor data associated with one or more food items in the food storage apparatus; process, by the computing device, the sensor data to identify the one or more food items and one or more attributes of the one or more food items; and determine, by the computing device, a restocking order of the one or more food items based on the one or more attributes of the one or more food items.

Example 47 may include the subject matter of Example 46, further comprising storing, by the apparatus, in a database, the determined restocking order.

Example 48 may include the subject matter of Example 46-47, further comprising: receiving, by the apparatus, a request for a recommendation; determining, by the apparatus, based upon the received request and the restocking order of the one or more food items, a recommendation; and transmitting, by the apparatus, the determined recommendation to a recipient.

Example 49 may include the subject matter of Example 48, wherein the recommendation is one or more of: which of the one or more food items to throw away; which of the one or more food items should be consumed within a first defined time period; which of the one or more food items should be consumed after a second defined time period; which of the one or more food items may be consumed within a third defined time period; which of the one or more food items should be sent to a first destination by a first date; or a best time to consume the one or more item of food.

Example 50 may include the subject matter of Example 48, wherein the recommendation is one or more of: what recipes may be made using the one or more food items; or what meals may be prepared with the one or more food items.

Example 51 may include the subject matter of Example 48, wherein the recommendation is which food items should be replenished and at what time.

Example 52 may include the subject matter of Example 48, wherein to determine a recommendation further comprises: to compare, by the computing device, the restocking order of the one or more food items with the stored restocking order of the one or more food items in the database.

Example 53 may include the subject matter of Example 52, wherein the recommendation is an indication of food items that have been consumed or are spoiled, based upon the comparison.

Example 54 may include the subject matter of any Examples 48-53, wherein to determine a recommendation further comprises to determine, by the computing device, a recommendation based on a predictive analytics engine.

Example 55 may include the subject matter of Example 48, wherein to transmit the determined recommendation further comprises to transmit or to cause to transmit a request for an action to a recipient, the action based upon the determined recommendation.

Example 56 may include the subject matter of Example 46, wherein a sensor is a camera, a 3D camera, or an infrared camera.

Example 57 may include the subject matter of Example 46, wherein a sensor is a thermometer, a humidity detector, a chemical analysis sensor, or an odor detector.

Example 58 may include the subject matter of Example 46, wherein a sensor is a mass sensor or a density sensor.

Example 59 may include the subject matter of Example 46, wherein a sensor is a softness sensor.

Example 60 may include the subject matter of any of Examples 46-59, wherein the computing device is included in a refrigerator, a freezer, a shipping container, a cooler, a pantry or a warehouse storage space.

What is claimed is:

1. An apparatus for storing food, comprising:
one or more sensors to collect sensor data associated with one or more food items;
one or more computer processors; and
a food condition module communicatively coupled to the one or more processors and the plurality of sensors to determine condition of the one or more food items;
wherein to determine, the food condition module is to:
process the sensor data to identify the one or more food items and one or more attributes of the one or more food items, and
determine a condition of the one or more food items based on the one or more attributes of the one or more food items.

2. The apparatus of claim 1, wherein the food condition module is to further store, in a database, the determined condition of the one or more food items.

3. The apparatus of claim 1, wherein the apparatus further includes:
a food recommendation module communicatively coupled to the one or more processors to provide a recommendation with respect to the identified one or more food items;
wherein to provide a recommendation, the food recommendation module is to:
receive a request for a recommendation;
determine, based upon the received request and the condition of the one or more food items, a recommendation; and
transmit the determined recommendation to a recipient.

4. The apparatus of claim 3, wherein the recommendation is one or more of:
which of the one or more food items to throw away;
which of the one or more food items should be consumed within a first defined time period;
which of the one or more food items should be consumed after a second defined time period;
which of the one or more food items may be consumed within a third defined time period;
which of the one or more food items should be sent to a first destination by a first date; or
a best time to consume the one or more item of food.

5. The apparatus of claim 3, wherein the recommendation is one or more of:
what recipes may be made using the one or more food items; or
what meals may be prepared with the one or more food items.

6. The apparatus of claim 3, wherein the recommendation is which food items should be replenished and at what time.

7. The apparatus of claim 3, wherein to determine a recommendation is further to:
compare the condition of the one or more food items with the stored condition of the one or more food items in a database.

8. The apparatus of claim 7, wherein the recommendation is an indication of consumed food, based upon the comparison.

9. The apparatus of claim 3, wherein to determine a recommendation is further to determine a recommendation based on a predictive analytics engine.

10. The apparatus of claim 3, wherein transmit the determined recommendation further comprises:
transmit or cause to transmit a request for an action, the action based upon the determined recommendation.

11. The apparatus of claim 1, wherein a sensor is a camera, a 3D camera, or an infrared camera.

12. The apparatus of claim 1, wherein a sensor is a thermometer, a humidity detector, a chemical analysis sensor, or an odor detector.

13. The apparatus of claim 1, wherein a sensor is a mass sensor or a density sensor.

14. The apparatus of claim 1, wherein a sensor is a softness sensor.

15. The apparatus of claim 1, wherein the apparatus is a refrigerator, a freezer, a shipping container, a cooler, a pantry or a warehouse storage space.

16. A method for storing food using a food storage apparatus comprising:
receiving, by the apparatus, sensor data associated with one or more food items in the food storage apparatus;
processing, by the apparatus, the sensor data to identify the one or more food items and one or more attributes of the one or more food items; and
determining, by the apparatus, an inventory of the one or more food items based on the one or more attributes of the one or more food items.

17. The method of claim 16, further comprising storing, by the apparatus, in a database, the determined inventory.

18. The method of claim 16, further comprising:
receiving, by the apparatus, a request for a recommendation;
determining, by the apparatus, based upon the received request and the inventory of the one or more food items, a recommendation; and
transmitting, by the apparatus, the determined recommendation to a recipient.

19. The method of claim 18, wherein the recommendation is one or more of:
what recipes may be made using the one or more food items; or
what meals may be prepared with the one or more food items.

20. An apparatus for storing food using a food storage apparatus comprising:
means for receiving sensor data associated with one or more food items in the food storage apparatus;
means for processing the sensor data to identify the one or more food items and one or more attributes of the one or more food items; and
means for determining menu opportunities using the one or more food items based on the one or more attributes of the one or more food items.

21. The apparatus of claim 20, further comprising storing, by the apparatus, in a database, the determined menu opportunities.

22. The apparatus of claim 20, further comprising:
means for receiving a request for a recommendation;
means for determining based upon the received request and the menu opportunities of the one or more food items, a recommendation; and
means for transmitting the determined recommendation to a recipient.

23. One or more computer readable media comprising instructions that cause a computing device, in response to execution of the instructions by the computing device, to:
receive, by the computing device, sensor data associated with one or more food items in the food storage apparatus;
process, by the computing device, the sensor data to identify the one or more food items and one or more attributes of the one or more food items; and
determine, by the computing device, a restocking order of the one or more food items based on the one or more attributes of the one or more food items.

24. The one or more computer readable media of claim 23, further comprising storing, by the apparatus, in a database, the determined restocking order.

25. The one or more computer readable media of claim 23, further comprising:
receiving, by the apparatus, a request for a recommendation;
determining, by the apparatus, based upon the received request and the restocking order of the one or more food items, a recommendation; and
transmitting, by the apparatus, the determined recommendation to a recipient.
